(19) United States
(12) Patent Application Publication
(10) Pub. No.: US 2006/0188887 A1
(43) Pub. Date: Aug. 24, 2006

(54) Method and system for elucidating the primary structure of biopolymers

(75) Inventors: Martin Bluggel, Dortmund (DE); Daniel Chamrad, Bochum (DE)

Correspondence Address: CONNOLLY BOVE LODGE & HUTZ, LLP, P O BOX 2207, WILMINGTON, DE, 19899, US

(73) Assignee: PROTAGEN AG, Dortmund (DE)

(21) Appl. No.: 10/557,501

(22) PCT Filed: May 24, 2004
(86) PCT No.: PCT/EP04/05548

§ 371 Date: Feb. 23, 2006

(30) Foreign Application Priority Data
May 23, 2003 (DE) 103239170

(51) Int. Cl. C12Q 1/68

(52) U.S. Cl. 435/6

(57) ABSTRACT

The present invention relates to a method and to a system (100) for predicting the primary structure of biopolymers, especially of proteins and peptides, in which at least two algorithms or the results of at least two algorithms and/or of bioinformatic analyses are combined in order to increase the significance of the results. The system (100) comprises a user interface (UI) for configuring and outputting the results as well as a database interface (DBI) with the databases (DB1, DB2) containing, for example, known amino acid sequences, and with which algorithms for bioinformatic sequences can be actuated.

The present invention relates to a method and to a system for predicting the primary structure of biopolymers, especially of proteins and peptides.

The computer-aided prediction of the structure of biopolymers using mass spectrometers is acquiring ever-greater significance.

The term “primary structure of biopolymers” refers to the chemical structure, especially to an appertaining sequence of the amino acids and their modifications such as, for instance, posttranslational modifications or chemical modifications.

Consequently, within the scope of this invention, the term “biopolymer” refers to a modified or unmodified polypeptide having at least one peptide bond and optionally non-protein fractions such as lip(o)ids, carbohydrates or other organic fractions and/or inorganic fractions such as metals.

The term “primary structure prediction” as employed here also refers to knowledge about errors in or deviations from existing sequence databases and modification databases as well as knowledge about single amino acid polymorphisms (SAPs).

The primary structure is normally predicted using mass spectrometric data. This mass spectrometric data is obtained by means of measurements using various known mass spectrometric methods.

In mass spectrometry (MS for short), suitable methods for biopolymers include electrospray mass spectrometry (ESI MS) and various methods of laser desorption such as, for instance, MALDI MS (see, in general, Budzikiewicz, Massenspektrometrie [Mass spectrometry], Weinheim, Germany (1998)).

In the description that follows, the term “mass spectrometric data” refers in particular to information about the molecular weight (or m/z value) of biopolymers or parts thereof (fragments) that are obtained through the targeted cleavage of one or more biopolymers.

In addition, before the biopolymers are cleaved, they can be modified specifically or non-specifically and the cleavage itself can likewise be carried out specifically, that is to say, it can be done at defined amino acids or else non-specifically, in other words, independently of specific amino acids.

The mass spectrometric data is evaluated by means of bioinformatic analyses, optionally employing a sequence database of known biopolymers and then, depending on the algorithm employed or on the bioinformatic analysis employed, conclusions can be drawn about the primary structure of the biopolymers or about the fragments of the biopolymers, for example, by making a comparison between the mass spectrometric data acquired through measurements and the data from the database.

Sequence databases contain either amino acid sequences of biopolymers or so-called genomic sequences from which the amino acid sequences can be derived.

When the primary structure of a biopolymers is predicted, it can happen that certain mass spectrometric data cannot be associated with any known data from the sequence database being used, so that the primary structure of an examined biopolymer can only be predicted partially or not at all.

Therefore, it is the objective of the present invention to improve a method or system of the generic type in such a way as to increase the significance of the results of the primary structure prediction, to render the prediction more complete, and also to simplify the method.

This objective is achieved according to the invention in that at least two algorithms or the results of at least two algorithms and/or of bioinformatic analyses are combined, as a consequence of which a total result can advantageously be derived that provides additional knowledge about the primary structure of the biopolymer and whose significance in terms of the possible primary structure of an examined biopolymer is greater than with the known methods.

A particularly advantageous approach is the combination according to the invention of so-called peptide mass fingerprint (PMF) algorithms and/or peptide fragmentation fingerprint (PFF) algorithms and/or algorithms from the family of the de novo sequencing algorithms and/or PTM prediction algorithms, all of which are known from the state of the art.

The PMF algorithm makes it possible to predict the primary structure of a polypeptide on the basis of an association of a measured mass spectrum with an entry in a sequence database. If the PMF algorithm cleaves the sequences of the database into peptides with the same specificity as the analyzed biopolymer had previously been cleaved into peptides, then a plurality of peptide sequences is obtained from which a theoretical mass spectrum can be created for each entry in the sequence database by means of the PMF algorithm.

Through a comparison of measured mass spectra with the theoretically determined mass spectra, a score can be assigned to each database entry on the basis of the result of this comparison, and this score reflects the degree of similarity between the mass spectra that have been compared. In the most favorable scenario, the particular database entry with the highest score matches the sequence of the analyzed biopolymer.

Analogously to the PMF algorithm, the PFF algorithm likewise employs sequence databases. Here, however, theoretical fragmentation spectra of peptides from the database are generated and compared to measured fragmentation data, on the basis of which—once again by evaluating the similarity—conclusions are drawn about a database entry.

The class of the de novo sequencing algorithms extracts information about the primary structure of the analyzed biopolymer directly from fragmentation spectra of peptides obtained through measurements made during the analysis of the biopolymers. In contrast to the PMF algorithms and PFF algorithms, the de novo sequencing algorithms do not employ any sequence databases.

The PTM prediction algorithm allows a prediction of posttranslational modifications and their position on the basis of the primary structure of the biopolymers, whereby information already known about posttranslational modifications and their positions within biopolymer sequences is utilized.

Experiments have shown that the combination of several of the cited algorithms markedly increases the significance in the prediction of the primary structure of an analyzed biopolymer. For example, the significance of a first result that is obtained using a first algorithm such as, for instance, the PMF algorithm, is markedly increased if the same result is also obtained when another algorithm is employed such as, for example, the PFF algorithm.

It is also possible to use two or more algorithms of the same type, in other words, for instance, two or more PFF algorithms. Owing to the different principles of operation of different algorithms of the same type, the significance of the results can likewise be increased in case of matching results and an improved prediction of the primary structure of the examined biopolymer can be attained.

A combination of several algorithms of the same type with one or more algorithms of a different type is likewise conceivable.

The method according to the invention is not restricted to the use of the algorithms cited; as an alternative or in addition to the cited algorithms, other algorithms can also be employed for mass spectrum analysis individually or in combination with each other, for example, for modification analysis and/or sequence error analysis and/or SAP (single amino acid polymorphism) algorithms and/or other algorithms.

A particularly advantageous variant of the method according to the invention is one in which information about the primary structure is obtained automatically from unpredicted fragmentation spectra, whereby specifiable chemical and posttranslational modifications and/or amino acid substitutions or other sequence errors and/or missing bonds are sought and/or whereby diverging ion masses are taken into consideration.

In this manner, mass spectra of a biopolymer that could not be associated at a sufficient significance with a known peptide or biopolymer during an analysis of its primary structure and subsequent evaluation by means of one or more algorithms, can be assigned a certain probability—taking into account possible modifications such as, for instance, posttranslational modifications or sequence errors or the like —with which these fragments match already known amino acid sequences.

In this context, a correlation between the unpredicted fragmentation spectra with known amino acid sequences is very advantageous.

According to another advantageous variant of the method, the unpredicted fragmentation spectra are correlated with other information about biopolymers in addition or as an alternative to the correlation with known amino acid sequences, whereby this other information is obtained from modification databases and/or from mass spectra databases and/or from nucleotide databases.

In a very advantageous manner, another embodiment of the method according to the invention provides for a storage of the results obtained by means of the above-mentioned correlation(s), so that the results can be used once again for future analyses, thus likewise contributing to improving the method and to increasing the significance of the results.

For example, during a subsequent analysis of a biopolymer, the stored results can already be incorporated into the prediction by means of the above-mentioned algorithms or combination of algorithms.

The use of a combination, that is to say, a plurality of fragmentation spectra for the analysis of an unpredicted fragmentation spectrum, is also particularly advantageous. For example, several fragmentation spectra can be obtained from the same sample of a biopolymer by means of several measurements which, for instance, due to imprecisions in the specificity of a cleavage of the biopolymer, yield different fragmentation spectra, both of which contain, for example, a cut-set of the amino acids that actually occur in the biopolymer. This translates into an improvement of the analysis results.

A system according to Patent Claim 10 is proposed as another way to achieve the objective of the present invention.

A particularly advantageous variant of the invention proposes an automatic acquisition of information about the primary structure of biopolymers from unpredicted fragmentation spectra of biopolymers so that fragmentation spectra that could only be associated partially or not at all with the primary structure known so far during a preceding analysis of the primary structure of a biopolymer can be assigned at least a certain probability with which these fragmentation spectra match a primary structure proposal, without a manual intermediate processing of the data.

An advantageous embodiment of the system according to the invention provides a user interface for entering parameters and/or for requesting results of bioinformatic analyses. As a result, a user of the system can control the course of the prediction of the primary structure of a biopolymer and can optionally request the results obtained.

The sequential control is effectuated, for example, through the selection of a number of parameters, each of which depends on the employed algorithms or bioinformatic analyses.

An advantageous embodiment of the user interface is an HTML interface (hypertext markup language interface) that can be implemented, for example, by a web server integrated into the system, which is available, for instance, as software for personal computers. Owing to the widespread availability of HTML-capable terminals, the system according to the invention can be accessed by numerous terminal devices such as, for example, notebooks or PDAs.

Other suitable interfaces are also a possibility instead of the HTML interface.

Another advantageous embodiment of the system according to the invention comprises a database interface that can access multiple databases. In this manner, for example, sequence databases or else databases in general can be accessed that contain results of bioinformatic analyses of biopolymers such as, for instance, mass spectrographic data.

The database interface according to the invention can likewise access modification databases and nucleotide databases as well as databases containing results of the above-mentioned correlation according to the invention with other information about biopolymers, whereby this other information, in turn, is obtained from modification databases and/or from mass spectra databases and/or from nucleotide databases.

In particular, the database interface of the system according to the invention also allows access to other bioinformatic systems which, for example, according to the algorithms known from the state of the art, carry out a correlation of unpredicted fragmentation spectra with known amino acid sequences.

According to another advantageous variant of the invention, the user interface has input and/or output masks for the employed algorithms in order to improve the general overview of the system.

In this context, when algorithms are used that simultaneously require largely the same or similar parameters, a common input mask is provided that can accept the same or similar parameters as well as parameters that are specific for each of the employed algorithms. As a result, the number of parameters that have to be provided redundantly for the employed algorithms is reduced and the user friendliness is enhanced.

Generally speaking, the system according to the invention can be implemented by a suitable sequential control, for instance, by means of a computer program that runs on a personal computer.

It is likewise very advantageous to use a system-internal database that stores, for example, (interim) results of bioinformatic analyses, parameters for algorithms as well as user-defined data. It is also very advantageous for the results of the above-mentioned correlation according to the invention of unpredicted fragmentation spectra to be stored with other information about biopolymers, whereby this other information, in turn, is obtained from modification databases and/or from mass spectra databases and/or from nucleotide databases. In this manner, the results can be re-used, for example, for a primary structure analysis.

The system-internal database can also be used to buffer data of external databases, thus enhancing the performance of the system.

Additional features, application possibilities and advantages of the invention can be gleaned from the description below of embodiments of the invention which are depicted in the figures in the drawing. In this context, all of the features described or depicted, either on their own or in any desired combination, constitute the subject matter of the invention, irrespective of the way in which they are compiled in the patent claims or the way in which they refer back thereto, as well as irrespective of their formulation or presentation in the description or in the drawing.

FIG. 1 schematically shows an embodiment of the system according to the invention;

FIG. 2 shows a screen view of an input mask of the user interface of the system according to the invention as shown in FIG. 1;

FIG. 3 shows a screen view of an output mask of the user interface as shown in FIG. 1; and

FIG. 4 shows a screen view of another input mask of the user interface as shown in FIG. 1.

FIG. 1 shows an embodiment of the system 100 according to the invention for predicting the primary structure of biopolymers, comprising a user interface UI and a database interface DBI.

The user interface UI serves to output data from the system 100 to a user and is implemented as an HTML interface. For this purpose, an integrated web server (not shown here) is provided in the system.

Moreover, via the HTML interface UI, which can be used via a web browser, the user can also make entries into the system 100, thus specifying, for example, parameters that are needed to run one or more algorithms that are used by the system 100 to predict the primary structure of biopolymers.

Such parameters can be stored in the internal database DB_100 of the system 100 so that they are available to be used again.

FIG. 2 shows an input mask that is provided by the user interface UI of the system 100 in order to configure the algorithms to be used.

In its upper left-hand area, the input mask has a selection field 210 where various algorithms for the analysis of a biopolymer can be selected.

Normally, mass spectrometric data of biopolymers or their fragments is transferred to the algorithms, on the basis of which matches between measured mass spectra and already known primary structures are then determined, optionally employing sequence tables or databases DB1, DB2 containing amino acid sequences of known biopolymers. The databases DB1, DB2 can also contain data other than sequence data, for instance, the databases DB1, DB2 can also be modification databases and/or mass spectra databases and/or nucleotide databases.

For this purpose, the system 100 is provided with a database interface DBI for accessing the databases DB1, DB2 and the algorithms A1, A2. The databases DB1, DB2 and the algorithms A1, A2 can communicate with each other. The databases DB1, DB2 are normally central or international databases that can be reached, for instance, via an Internet connection. In addition or as an alternative, information or amino acid sequences and the like that are stored in the internal database DB_100 can also be accessed.

In particular, the internal database DB_100 also contains results from correlations in which unpredicted fragmentation spectra of analyzed biopolymers have been correlated with information from modification databases and/or from mass spectra databases and/or from nucleotide databases. These results can be further employed for future analyses or made available to external systems.

The database interface DBI converts user entries from the user interface UI or data from the internal database DB_100 into the format needed for the algorithms A1, A2. It is also possible to uniformly present data such as, for instance, parameters or results, etc. internally in the system 100, for example, by means of XML (extensible markup language) and, whenever needed, for example, in order to exchange data with other systems, to convert it from the XML format into the necessary target format.

The upper right-hand area of FIG. 2 shows another field 220 of the input mask that serves to configure parameters that are needed by the algorithms to be employed or by the databases DB1, DB2 (FIG. 1) necessary for this purpose.

Finally, the lower part of the input mask shows a parameter field 230 that serves for the manipulation of individual parameters of the algorithms employed, and also a button which, when activated, starts the analysis by means of the selected algorithms.

An output mask depicted in FIG. 3 shows such a result of the analysis, said mask containing partial results of the analysis listed in tabular form. In this context, a score obtained by means of the first algorithm selected for the analysis is entered in column 305, while a score obtained by means of the second algorithm selected for the analysis is entered in column 306.

Each of these scores is a measure of the match between measured mass spectrometric data of the analyzed biopolymer or its fragments and the already known amino acid sequences found in the databases DB1, DB2.

In addition, column 300 also shows a characteristic number designated as a “MetaScore”, which is ascertained by means of a specific method from a combination of the results of both of the employed algorithms and which has a considerably higher significance in comparison to the scores of columns 305 and 306.

Therefore, a more reliable analysis of the biopolymer is possible in comparison to conventional methods.

Another input mask to control another algorithm for predicting the primary structure of biopolymers can be seen in FIG. 4.

All in all, for each algorithm implemented in the system 100 or supported by the system 100, a special input mask is provided in order to ensure user friendliness, or else different algorithms, especially those that require similar parameters or even a plurality identical parameters, are controlled by means of a shared input mask.

Examples of suitable algorithms for the analysis are a peptide mass fingerprint (PMF) algorithm and/or a peptide fragmentation fingerprint (PFF) algorithm and/or an algorithm from the family of the de novo sequencing algorithms and/or a PTM prediction algorithm and/or another algorithm for the mass spectrometric or modification analysis. By the same token, it is also conceivable to employ several algorithms of the same type, thus, for example, two PMF algorithms or PFF algorithms, or else a combination of several algorithms of the same type as well as the other above-mentioned algorithms.

Should additional algorithms become available, their use can be made possible by implementing an appropriate input mask and a corresponding output mask.

In addition to the input and output masks of the user interface UI, the system 100 also comprises elements (not shown here) for sequential control which are partially algorithm-specific, that is to say, provided for the specific control of the individual algorithms.

A particular advantage of the present invention lies in the fact that unpredicted fragmentation spectra of analyzed biopolymers are automatically compared to a primary structure proposal.

For this purpose, specifiable chemical and posttranslational modifications and/or amino acid substitutions or other sequence errors and/or missing bonds are sought and/or diverging ion masses are taken into consideration.

The unpredicted fragmentation spectra can also be correlated with known amino acid sequences, especially from sequence databases or, as already mentioned, with other primary structure data from databases.

By the same token, the primary structure prediction can be improved by combining several fragmentation spectra.

To this end, analogously to the analyses already described, corresponding algorithms are activated or database searches are started in the databases DB1, DB2 by means of an appropriate sequential control unit (not shown here) in the system 100.

The results are once again displayed in an appropriate output mask.

The system 100 can be installed, for example, on a personal computer with the appropriate program controls. It is, however, also possible to distribute individual analyses or database accesses over several systems 100 in order to enhance the system performance that can be achieved. In this case, it is advantageous if each system can access the results of the other systems.

Generally speaking, the method according to the invention can be used to predict parts of the primary structure of a biopolymer or even the entire primary structure, whereby, for example, interim results obtained when parts are predicted can be stored and thus made available for future analyses.

1. A method for predicting the primary structure of biopolymers by means of mass spectrometric data in which at least two algorithms or the results of at least two algorithms and/or bioinformatic analyses are combined.

2. The method according to claim 1, characterized in that algorithms for modification analysis and/or sequence error analysis and/or SAP (single amino acid polymorphism) algorithms and/or algorithms for mass spectrum analysis are employed.

3. The method according to claim 1, characterized in that peptide mass fingerprint (PMF) algorithms and/or peptide fragmentation fingerprint (PFF) algorithms and/or algorithms from the family of the de novo sequencing algorithms and/or PTM prediction algorithms are employed as algorithms.

4. The method according to claim 1, characterized in that at least two algorithms of the same type are employed, especially at least two peptide mass fingerprint (PMF) algorithms and/or at least two peptide fragmentation fingerprint (PFF) algorithms and/or at least two algorithms from the family of the de novo sequencing algorithms.

5. The method according to claim 1, characterized in that information about the primary structure is obtained automatically from unpredicted fragmentation spectra, whereby specifiable chemical and posttranslational modifications and/or amino acid substitutions or other sequence errors and/or missing bonds are sought and/or whereby diverging ion masses are taken into consideration.

6. The method according to claim 1, characterized in that unpredicted fragmentation spectra are correlated with known sequences, especially from sequence databases and/or with other information about biopolymers, whereby the other information can be obtained from modification databases and/or from mass spectra databases.

7. The method according to claim 6, characterized in that the results of the correlation are stored.

8. The method according to claim 7, characterized in that the stored results are employed for predicting the primary structure of biopolymers.

9. The method according to claim 1, characterized in that unpredicted fragmentation spectra are analyzed using a combination of fragmentation spectra.

10. A system (100) for predicting the primary structure of biopolymers by means of mass spectrometric data in which at least two algorithms or the results of at least two algorithms and/or of bioinformatic analyses can be combined.

11. The system (100) according to claim 10, characterized in that information about the primary structure of biopolymers can be obtained automatically from unpredicted fragmentation spectra.

12. The system (100) according to claim 10, characterized in that a user interface (UI) is provided for entering parameters and/or for requesting results of bioinformatic analyses, especially of unpredicted fragmentation spectra.

13. The system (100) according to claim 12, characterized in that the user interface (UI) is an HTML interface.

14. The system (100) according to claim 10, characterized in that a database interface (DBI) is provided for accessing a plurality of databases (DB1, DB2), especially sequential databases and/or databases with mass spectra and/or modification databases.

15. The system (100) according to claim 10, characterized in that the user interface (UI) has input and/or output masks for the employed algorithms (A1, A2, etc.).

16. The system (100) according to claim 10, characterized by at least one database (DB_100).

17. The system (100) according to claim 10, which is suitable for carrying out a method for predicting the primary structure of biopolymers by means of mass spectrometric data in which at least two algorithms or the results of at least two algorithms and/or bioinformatic analyses are combined.
